(19) United States
(12) Patent Application Publication
(10) Pub. No.: US 2017/0081624 A1
(43) Pub. Date: Mar. 23, 2017

(54) PACKAGING BAG FOR CELL CULTURE VESSEL

(71) Applicant: SUMITOMO BAKELITE CO., LTD., Shinagawa-ku (JP)

(72) Inventor: Ryohei TSUKADA, Tokyo (JP)

(73) Assignee: SUMITOMO BAKELITE CO., LTD., Shinagawa-ku (JP)

(21) Appl. No.: 15/265,035

(22) Filed: Sep. 14, 2016

(30) Foreign Application Priority Data
Sep. 17, 2015 (JP) 2015-184327

(51) Int. Cl. C12M 1/00; C12M 1/12

(52) U.S. Cl. CPC C12M 23/14; C12M 37/04; C12M 47/16

(57) ABSTRACT

A packaging bag for a cell culture vessel used for sealing a cell culture vessel, wherein when the packaging bag for a cell culture vessel sealing the cell culture vessel is accommodated in a closed space in which ordinary pressure is maintained, a temperature of 45° C. is maintained and the volume is 170 L, and the total amount of 70 mL of 6-mass % hydrogen peroxide aqueous solution is vaporized by applying ultrasonic vibration for 7 minutes in the closed space, and the hydrogen peroxide is decomposed by ultraviolet irradiation at a wavelength of 254 nm so that hydrogen peroxide concentration in the closed space becomes equal to or smaller than 1 mg/L within 90 minutes from the stop of the ultrasonic vibration, the amount of hydrogen peroxide adsorbed to the cell culture vessel is less than 3 ng/cm².

### BACKGROUND OF THE INVENTION

Field of the Invention

The present disclosure relates to a packaging bag for a cell culture vessel, a package formed by packing a cell culture vessel in the packaging bag for a cell culture vessel, a manufacturing method of the package, and a decontaminating method of the package. The present application claims priority to Japanese Patent Application No. 2015-184327 filed on Sep. 17, 2015 and the contents of which is incorporated herein by reference.

Description of Related Art

A culturing operation of stem cells or cell aggregation such as human embryonic stem cells (human ES cells) or human pluripotent stem cells (human iPS cells) is performed in a highly sterile environment. Specifically, after putting a packaged culture vessel in an isolator, for example, a pollution source of germs or the like which may enter the isolator in accordance with the input of the culture vessel into the isolator, is decontaminated by using a treating agent such as hydrogen peroxide gas or the like. After that, aeration is performed to set hydrogen peroxide concentration in the isolator to be equal to or less than a predetermined value, and then, the culture vessel is extracted from the packaging bag.

Patent Document 1 discloses an example of a cell culture vessel used in the culturing of ES cells or iPS cells.

[Patent Document 1] Japanese Unexamined Patent Application, First Publication No. 2015-065942

However, hydrogen peroxide which has transmitted a packaging material packing a culture vessel during the decontamination and is absorbed to the culture vessel, is melted in a culture medium to be supplied to the culture vessel and the hydrogen peroxide in the culture medium may negatively affect growth of cells or cell aggregation.

Therefore, an object of the disclosure is to provide a packaging bag for a cell culture vessel having high hydrogen peroxide barrier properties and capable of preventing negative effects of hydrogen peroxide to productivity of cells or cell aggregation, a package formed by packing a culture vessel in the packaging bag for a cell culture vessel, and a manufacturing method of the package.

### SUMMARY OF THE INVENTION

According to one or plural aspects of the present disclosure, there is provided a packaging bag for a cell culture vessel used for sealing a cell culture vessel, in which an amount of hydrogen peroxide adsorbed to the cell culture vessel is less than 3 ng/cm² in a case where the packaging bag for a cell culture vessel scaling the cell culture vessel is treated under the following conditions. In the treatment described above, the total amount of 70 mL of 6-mass % hydrogen peroxide aqueous solution is vaporized by applying ultrasonic vibration for 7 minutes in a closed space in which ordinary pressure is maintained, a temperature is maintained at 45° C., volume is 170 L, and the packaging bag for a cell culture vessel sealing the cell culture vessel is accommodated, to set hydrogen peroxide vapor, and hydrogen peroxide is decomposed by ultraviolet irradiation at a wavelength of 254 nm so that hydrogen peroxide concentration in the closed space becomes equal to or smaller than 1 mg/L within 90 minutes from the stop of the ultrasonic vibration.

According to one or plural aspects of the present disclosure, there is provided a package including: the packaging bag for a cell culture vessel of the disclosure; and a cell culture vessel sealed in the packaging bag for a cell culture vessel.

According to one or plural aspects of the present disclosure, there is provided a manufacturing method of a package including: a step of sealing a cell culture vessel in the packaging bag for a cell culture vessel of the disclosure; and a step of sterilization step of performing radiation sterilization of the packaging bag for a cell culture vessel sealing the cell culture vessel therein.

According to the disclosure, it is possible to provide a packaging bag for a cell culture vessel having high hydrogen peroxide barrier properties and capable of preventing negative effects of hydrogen peroxide to productivity of cells or cell aggregation, a package formed by packing a culture vessel in the packaging bag, and a manufacturing method of the package.

### BRIEF DESCRIPTION OF THE DRAWINGS

FIG. 1 is a plan schematic view showing an example of a packaging bag for a cell culture vessel of the disclosure and a package of the disclosure.

FIG. 2 is a plan schematic view showing another example of the packaging bag for a cell culture vessel of the disclosure and another example of the package of the disclosure.

FIG. 3 is an enlarged sectional view showing an example of a packaging material of the packaging bag for a cell culture vessel of the disclosure.

FIG. 4 is an enlarged sectional view showing another example of the packaging material of the packaging bag for a cell culture vessel of the disclosure.

FIG. 5 is an enlarged sectional view showing another example of the packaging material of the packaging bag for a cell culture vessel of the disclosure.

FIG. 6 is a plan schematic view showing another example of the packaging bag for a cell culture vessel of the disclosure and another example of the package of the disclosure.

### DETAILED DESCRIPTION OF THE INVENTION

According to one embodiment, the present disclosure relates to a packaging bag for a cell culture vessel used for sealing a cell culture vessel, (hereinafter, may be abbreviated as a “packaging bag”), in which an amount of hydrogen peroxide adsorbed to the cell culture vessel is less than 3 ng/cm² in a case where the packaging bag sealing the cell culture vessel is set as a target of the treatment under the following conditions. The amount of hydrogen peroxide adsorbed to the cell culture vessel is preferably equal to or smaller than 1 ng/cm², in order to prevent negative effects of hydrogen peroxide to productivity of cells or cell aggregation. In a case where the treatment under the following conditions is performed, when the cell culture vessel is packaged by using a packaging bag in which the amount of hydrogen peroxide adsorbed to the cell culture vessel is less than 3 ng/cm², it is possible to prevent negative effects of hydrogen peroxide to productivity of cells or cell aggregation.

In the treatment, the total amount of 70 mL of 6-mass % hydrogen peroxide aqueous solution is vaporized by applying ultrasonic vibration for 7 minutes in the closed space in which ordinary pressure is maintained, a temperature is maintained at 45° C., volume is 170 L, and the packaging bag sealing the cell culture vessel is accommodated, to set hydrogen peroxide vapor. Accordingly, the packaging bag sealing the cell culture vessel is exposed to hydrogen peroxide vapor and hydrogen peroxide is decomposed by ultraviolet irradiation at a wavelength of 254 nm so that hydrogen peroxide concentration in the closed space becomes equal to or smaller than 1 mg/L within 90 minutes from the stop of the ultrasonic vibration. The ordinary pressure means pressure equal to atmospheric pressure and is substantially one atmosphere.

In the disclosure, the “amount (ng/cm²) of hydrogen peroxide adsorbed to a cell culture vessel” is obtained by extracting a cell culture vessel from a packaging bag which is a target of the treatment, rapidly dissolving hydrogen peroxide adsorbed to the cell culture vessel in ultrapure water, measuring concentration of hydrogen peroxide in the dissolved solution, and converting the concentration into an amount thereof adsorbed per unit area. The concentration of hydrogen peroxide in the dissolved solution is determined from absorbance of light at 550 nm of the dissolved solution including an enzyme and a color former, and a calibration curve which is previously created. Peroxidase disclosed in the example is used, for example, as an enzyme and 4-aminoantipyrine disclosed in the example is used, for example, as a color former. A calculating method of the “amount (ng/cm²) of hydrogen peroxide adsorbed to a cell culture vessel” will be specifically described in the example.

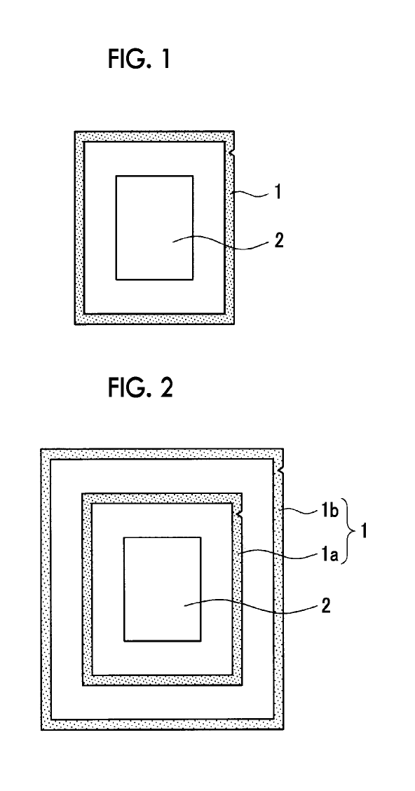

The packaging bag of the disclosure is, for example, a flexible packaging bag formed by providing heat sealing portions on two or three sides of the periphery of a double-folded or two-sheet laminated packaging material and forming an opening portion on one side. A packaging bag 1 of the disclosure may be a single-layered packaging bag which covers a cell culture vessel 2 with a single-layered packaging material in accordance with characteristics of a packaging material configuring a packaging bag (see FIG. 1), or may be a double-layered packaging bag which includes an inner bag 1a and an outer bag 2b and covers the cell culture vessel 2 with a double-layered packaging material (see FIG. 2). In a case where the packaging bag 1 of the disclosure is a multiple-layered packaging bag, the structure of the packaging material covering the cell culture vessel is not limited to double-layered structure and may be triple-layered or higher structure, and is preferably triple-layered or lower structure, in order to improve operability of opening and preventing generation of dust at the time of operating the opening.

The cell culture vessel is put into the packaging bag of the disclosure and the opening portion is heat-sealed. Accordingly, an example of the package of the disclosure including the packaging bag and the cell culture vessel scaled in the packaging bag (see FIG. 1) is obtained. In a case where the packaging bag of the disclosure is a multiple-layered packaging bag, the cell culture vessel is put into a bag on the innermost side configuring the multiple-layered packaging bag and the opening portion of the bag is heat-sealed to seal the cell culture vessel in the bag. Then, the bag containing the cell culture vessel is sealed in another bag configuring the multiple-layered packaging bag, and this operation is repeated by the number of times of the number of the remaining bags configuring the multiple-layered packaging bag. Accordingly, another example of the package of the disclosure (see FIG. 2) is obtained. The packaging bag of the disclosure and each bag configuring the packaging bag of the disclosure may include a V-notch opening slit.

The structure of the cell culture vessel sealed in the packaging bag of the disclosure or the cell culture vessel configuring the package of the disclosure is not particularly limited and a cell culture vessel including a petri dish or a plurality of wells is used, for example. The packaging bag of the disclosure is suitable for the packaging of a cell culture vessel which is used for long-term cell culture, easily receives negative effects of hydrogen peroxide, and is used for culturing stem cells or cell aggregation such as human embryonic stem cells (human ES cell) or human pluripotent stem cells (human iPS cells), among various cell culture vessels.

The packaging bag of the disclosure is set as a target of sterilization, is preferably set as a target of radiation sterilization, and more preferably set as a target of electron beam sterilization.

Packaging Material

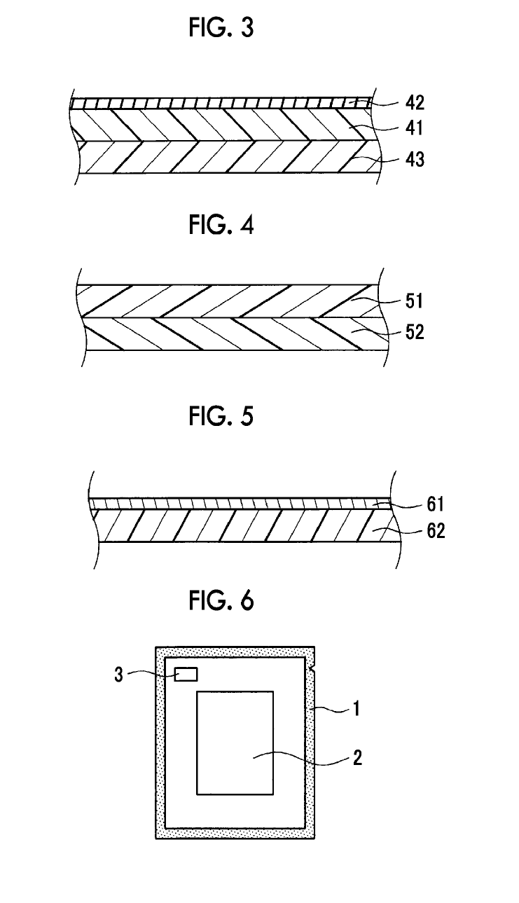

In one or plural embodiments, a packaging material of the packaging bag according to the disclosure is preferably at least one type selected from a laminated film A (see FIG. 3) including a base film, an inorganic thin film layer 42 provided on one surface side of the base film 41, and a sealant layer 43 provided on another surface side of the base film 41, a laminated film B (see FIG. 4) including a base film 51, and a sealant layer 52 provided on one surface side of the base film 51, and a laminated film C (see FIG. 5) including a metal layer 61, and a sealant layer 62 provided on one surface side of the metal layer 61, and the packaging material is more preferably the laminated film A or the laminated film B having high visibility of the inside of the packaging bag, or the laminated film A or the laminated film C, from a viewpoint of improving hydrogen peroxide barrier properties, and even more preferably the laminated film A.

A thickness of the packaging material of the packaging bag according to the disclosure is preferably equal to or greater than 10 μm and more preferably equal to or greater than 30 μm, from a viewpoint of maintaining strength, and is preferably equal to or smaller than 150 μm or more preferably equal to or smaller than 120 μm, from a viewpoint of bending resistance.

Laminated Film A

As shown in FIG. 3, the laminated film A, for example, includes the base film 41, the inorganic thin film layer 42 provided on one surface of the base film 41, and the sealant layer 43 provided on another surface side of the base film 41.

Specific examples of the base film 41 configuring the laminated film A include films using polyester such as polyethylene, polyolefin such as polypropylene, polyethylene terephthalate, or polyethylene-2,6-naphthalate, polyamide such as Nylon 6, Nylon 66, or Nylon 12, polyimide, polyether imide, polycarbonate (PC), polyvinyl butyral, or a fluorine resin as a raw material. Among these, polyester is more preferable and polyethylene terephthalate is more preferable, from viewpoints of high transparency and low hygroscopicity, and polyolefin is preferable from a viewpoint of high flexibility. The base film 41 is preferably a multilayer film obtained by laminating two or more layers of films using the resin as a raw material, and is more preferably a laminate of polyolefin films.

The base film 41 can be manufactured by using a well-known general method in the related art. The base film 41 may be any of an unstretched film, a uniaxially oriented film, and a biaxial oriented film, and is preferably a uniaxially oriented film or a biaxial oriented film and more preferably a biaxial oriented film, from a viewpoint of high barrier properties.

A thickness of the base film 41 is preferably from 10 to 150 μm and more preferably from 10 to 100 μm, from viewpoints of high mechanical strength, high flexibility, and high transparency.

Examples of an inorganic material configuring the inorganic thin film layer 42 include oxides, carbides, or nitrides of silicon, aluminum, magnesium, zinc, tin, nickel, or titanium, and is preferably one kind or two or more kinds selected from the group consisting of silicon oxide, silicon carbide, silicon nitride, aluminum oxide, aluminum carbide, and aluminum nitride and more preferably an element containing silicon oxide, from viewpoints of high transparency and high hydrogen peroxide barrier properties. As a forming method of the inorganic thin film layer 42, a vapor deposition method is preferable, in order to obtain an even thin film having high gas barrier properties. Examples of the vapor deposition method include a chemical vapor deposition method (CVD method), ion plating, and sputtering.

A thickness of the inorganic thin film layer 42 is preferably equal to or greater than 5 nm and more preferably equal to or greater than 10 nm, from a viewpoint of obtaining high gas barrier properties, and is preferably equal to or smaller than 400 nm and more preferably equal to or smaller than 100 nm, from a viewpoint of preventing fracture at the time of bending.

The sealant layer 43 is one outermost layer of the laminated film A, is a heat-sealing resin layer which is melted by heat, and is also referred to as a heat-sealing layer. Specific examples of the sealant layer 43 include resin films of polyethylene, polypropylene, an ethylene-vinyl acetate copolymer, an ethylene-acrylic acid copolymer, and an ethylene-methacrylic acid copolymer. As a method of laminating the sealant layer 43 on the base film 41, an extrusion lamination method of the related art or a dry lamination method is used.

From a viewpoint of improving adhesiveness of the inorganic thin film layer 42, an anchor coating agent is preferably applied to a surface of a lower layer where the inorganic thin film layer 42 is formed, for example, a surface of the base film 41 where the inorganic thin film layer 42 is formed. As an anchor coating agent, a polyester resin, an acrylic resin, and an ethylene vinyl alcohol resin can be used alone or a mixture of two or more kinds thereof.

Atop coat layer may be formed on the inorganic thin film layer 42. As a top coating agent, a polyester resin, an acrylic resin, and an ethylene vinyl alcohol resin can be used alone or a mixture of two or more kinds thereof. As a forming method of the top coat layer 44, an extrusion lamination method of the related art or a dry lamination method is used.

In the laminated film A, as an example of a combination of the sealant layer/base film/inorganic thin film layer, polyethylene/polyethylene terephthalate/silicon oxide, polyethylene/polyamide/silicon oxide, polyethylene/polypropylene/silicon oxide, polyethylene/polyethylene terephthalate/aluminum oxide, polyethylene/polyamide/aluminum oxide, and polyethylene/polypropylene/aluminum oxide are preferable, and polyethylene/polyethylene terephthalate/silicon oxide or polyethylene/polyethylene terephthalate/aluminum oxide is more preferable, from viewpoints of high transparency and high hydrogen peroxide barrier properties.

Vapor permeability of the laminated film A measured at 40° C. and 90% RH (relative humidity) is preferably small as possible, from a viewpoint of improving hydrogen peroxide barrier properties, and is preferably equal to or smaller than 0.2 g/(m²·24 hr) and more preferably equal to or smaller than 0.1 g/(m²·24 hr). Vapor permeability of the laminated film A can be measured based on JIS Z0222.

Oxygen permeability of the laminated film A measured at 25° C. and 80% RH (relative humidity) is preferably small as possible, from a viewpoint of improving hydrogen peroxide harrier properties, and is preferably equal to or smaller than 2.0 mL/(m²·24 hr·MPa) and more preferably equal to or smaller than 1.5 mL/(m²·24 hr·MPa). Oxygen permeability of the laminated film A can be measured based on JIS K7126-2. JIS K7126-2 corresponds to ISO 15105-2.

The packaging bag of the disclosure formed by using the laminated film A may be a single-layered packaging bag or may be a multiple-layered packaging bag.

Laminated Film B

As shown in FIG. 4, the laminated film B includes the base film 51 and the sealant layer 52 provided on one surface side of the base film. The raw material and the manufacturing method of the base film 51 configuring the laminated film B may be the same as those of the base film 41 configuring the laminated film A, but a thickness of the base film 51 configuring the laminated film B is preferably from 30 to 150 μm and more preferably from 50 to 120 μm, from a viewpoint of mechanical strength of the packaging bag. It is preferable to perform a well-known process for easy tearing with respect to the base film 51 configuring the laminated film B, from a viewpoint of improving openability of the packaging bag.

The raw material, the manufacturing method, and the thickness of the sealant layer 52 configuring the laminated film B may be the same as those of the sealant layer 43 configuring the laminated film A.

A preferable layer configuration (sealant layer/base film) of the laminated film B is preferably a heat sealing polyolefin resin/polyester or a heat sealing polyolefin resin/polyamide/polyolefin resin, from a viewpoint of improving hydrogen peroxide barrier properties, more preferably polyethylene/polyethylene terephthalate, or polyethylene/nylon/polyethylene, and even more preferably polyethylene/nylon/polyethylene, from a viewpoint of improving hydrogen peroxide barrier properties. From a viewpoint of improving hydrogen peroxide barrier properties and improving openability of the packaging bag, the layer structure thereof is preferably heat sealing polyolefin resin/polyester subjected to the process for easy tearing, or heat sealing polyolefin resin/polyamide subjected to the process for easy tearing/polyethylene, more preferably polyethylene/polyethylene terephthalate or polyethylene/nylon subjected to the process for easy tearing/polyethylene, and even more preferably heat sealing polyolefin/nylon subjected to the process for easy tearing/polyethylene.

Vapor permeability of the laminated film B measured under the measurement conditions of 40° C. and 90% RH (relative humidity) is preferably small as possible, from a viewpoint of improving hydrogen peroxide barrier properties, and is preferably equal to or smaller than 10 g/(m²·24 hr) and more preferably equal to or smaller than 5 g/(m²·24 hr). Vapor permeability of the laminated film B can be measured based on JIS Z0222.

Oxygen permeability of the laminated film B measured under the measurement conditions of 25° C. and 80% RH (relative humidity) is preferably small as possible, from a viewpoint of improving hydrogen peroxide barrier properties, and is preferably equal to or smaller than 10000 mL/(m²·24 hr·MPa) and more preferably equal to or smaller than 5000 mL/(m²·24 hr·MPa). Oxygen permeability of the laminated film B can be measured based on JIS K7126-2. JIS K7126-2 corresponds to ISO 15105-2.

The packaging bag of the disclosure formed by using the laminated film B is preferably a double-layered packaging bag or a triple-layered packaging bag, from a viewpoint of improving hydrogen peroxide barrier properties and improving handleability of the package.

Laminated Film C

As shown in FIG. 5, the laminated film C includes the metal layer 61 and the sealant layer 62 provided on one surface side of the metal layer 61. The metal layer 61 may be any of a metal foil or a metal deposited layer. Examples of a forming method of the metal layer 61 include well-known vacuum deposition of the related art, sputtering or lamination of metal foil. A thickness of the metal layer 61 is preferably from 0.01 to 50 μm and more preferably from 0.1 to 30 μm, from viewpoints of improving hydrogen peroxide barrier properties and mechanical strength of the packaging bag.

The raw material, the manufacturing method, and the thickness of the sealant layer 62 configuring the laminated film C may be the same as those of the sealant layer 43 configuring the laminated film A.

As a metal material configuring the metal layer 61, one kind or two or more kinds selected from the group consisting of silver, copper, aluminum, titanium, chromium, manganese, iron, nickel, zinc, and stainless steel, and aluminum is particularly preferably used from viewpoints of hydrogen peroxide barrier properties, availability, and price.

Atop coat layer may be formed on the metal layer 61. The raw material, the manufacturing method, and the thickness of the top coat layer configuring the laminated film C may be the same as the top coat layer configuring the laminated film A.

A preferable layer configuration of the laminated film C is preferably heat sealing polyolefin resin/metal layer/polyolefin resin or heat sealing polyolefin resin/metal layer/polyolefin resin/polyamide, and more preferably heat sealing polyethylene/aluminum layer/polyethylene or heat sealing polyethylene/aluminum layer/polyethylene/nylon, from a viewpoint of improving hydrogen peroxide barrier properties.

Vapor permeability of the laminated film C measured under the measurement conditions of 40° C. and 90% RH (relative humidity) is preferably small as possible, from a viewpoint of improving hydrogen peroxide barrier properties, and is preferably equal to or smaller than 0.2 g/(m²·24 hr) and more preferably equal to or smaller than 0.1 g/(m²·24 hr). Vapor permeability of the laminated film C can be measured based on JIS Z0222.

Oxygen permeability of the laminated film C measured under the measurement conditions of 25° C. and 80% RH (relative humidity) is preferably small as possible, from a viewpoint of improving hydrogen peroxide barrier properties, and is preferably equal to or smaller than 2.0 mL/(m²·24 hr·MPa) and more preferably equal to or smaller than 1.5 mL/(m²·24 hr·MPa). Oxygen permeability of the laminated film C can be measured based on JIS K7126-2. JIS K7126-2 corresponds to ISO 15105-2.

The packaging bag of the disclosure formed by using the laminated film C may be a single-layered packaging bag or may be a multiple-layered packaging bag.

The packaging material of the packaging bag according to the disclosure is not limited to the laminated film A, the laminated film B, and the laminated film C. The packaging material of the packaging bag according to the disclosure may be a laminated film obtained by laminating two or more layers selected from a layer configuring the laminated film A, a layer configuring the laminated film B, and a layer configuring the laminated film C, and may be a laminated film including both of an inorganic thin film and a metal layer and a laminated film including both of a silicon oxide film and an aluminum layer, from a viewpoint of improving gas barrier properties, for example.

In a case where the packaging bag according to the disclosure is a multiple-layered packaging bag, the packaging materials configuring each bag may be the same as each other or may be different from each other.

The packaging bag of the disclosure described with reference to FIG. 1 and FIG. 2 has a flat bag shape, but the packaging bag of the disclosure may be a bag with gusset.

In a case where the packaging bag of the disclosure or the package of the disclosure is subjected to radiation sterilization, particularly, γ ray sterilization, in the packaging bag of the disclosure or the package of the disclosure, it is preferable that the bag is formed with the packaging material having high gas barrier properties and a gas absorber 3 (sec FIG. 6) is provided in the bag.

When the gas absorber is sealed in the packaging bag together with the cell culture vessel, oxidizing gas generated from the cell culture vessel by performing γ ray sterilization is absorbed to the gas absorber and negative effects of the oxidizing gas affected to cells or cell aggregation are decreased. In a case where the packaging bag according to the disclosure is a multiple-layered packaging bag, it is preferable that the gas absorber is sealed in a bag on the innermost side. It is assumed that the oxidizing gas contains any of alcohol, aldehyde, ketones, and organic acid.

As a specific example of the gas absorber, a resin film obtained by kneading an absorbing agent (for example, activated carbon or zeolite) of the oxidizing gas is used, and OxyCatch (registered trademark, manufactured by Kyodo Printing Co., Ltd.) is used as a commercially available product.

Sterilization

According to one embodiment, the package of the disclosure is a target of sterilization, preferably a target of radiation sterilization, and more preferably a target of electron beam sterilization, from a viewpoint of preventing generation of oxidizing gas.

A dose rate of radiation in a case of performing radiation sterilization is equal to or greater than 0.1 kGy/hr, preferably equal to or greater than 1 kGy/hr, more preferably equal to or greater than 10 kGy/hr, and even more preferably equal to or greater than 100 kGy/hr, from a viewpoint of preventing the amount of oxidizing gas generated from the packaging bag and the vessel for culture, and is preferably equal to or smaller than 100 MGy/hr, more preferably equal to or smaller than 10 MGy/hr, and even more preferably equal to or smaller than 1 MGy/hr, from a viewpoint of preventing heat generation. In the electron beam sterilization, a dose rate of radiation is generally preferably from 100 kGy/hr to 100 MGy/hr. In γ ray sterilization, a dose rate of radiation is generally preferably from 0.1 kGy/hr to 100 kGy/hr.

An amount of irradiated radiation of the radiation is determined by using any method disclosed in JIS-T0806, in accordance with an abacterial level required to the package. The amount of irradiated radiation of the radiation means absorbed dose of cumulative radiation of the package.

A material of the cell culture vessel is not particularly limited. The cell culture vessel can be set as a disposal type, and a resin is preferably used, from a viewpoint of easy moldability. Examples of the resin include a polyolefin-based resin or a cyclic polyolefin-based resin such as a polypropylene resin, a polyethylene resin, and an ethylene-propylene copolymer, a polystyrene-based resin such as polystyrene, an acrylonitrile-butadiene-styrene-based resin, a methacrylic resin such as a polycarbonate resin, a polyethylene terephthalate resin, or a polymethyl methacrylate resin, a fluorine-based resin such as a vinyl chloride resin, a polybutylene terephthalate resin, a polyarylate resin, a polysulfone resin, a polyether sulfone resin, a polyether etherketone resin, a polyether imide resin, or polytetrafluoroethylene, an acrylic resin such as polymethylpentene resin or polyacrylonitrile, and a cellulose-based resin such as a propionate resin. Among these, a polystyrene resin is preferable, from viewpoints of moldability required to the cell culture vessel and sterilization properties.

The shape of the cell culture vessel is not particularly limited, and a petri dish (dish), a multi-well plate including a plurality of wells, a flask, or a tube is used, and among these, a multi-well plate or a petri dish used for generation of bioreactor or evaluation of drug effect or toxic materials and development research of artificial organs is preferable. The multi-well plate is a substrate including a plurality of recesses opened on the upper surface. The number of wells of the multi-well plate is not particularly limited, and is, for example, 6, 12, 24, 48, 96, or 384. The multi-well plate may be, for example, a multi-well plate disclosed in Japanese Unexamined Patent Application, First Publication No. 2013-70636 and Japanese Unexamined Patent Application, First Publication No. 2012-210166.

Decontaminating Method

In one or plural embodiments, a method of decontaminating the package of the disclosure is provided. The decontaminating method is any of a method of dipping the package of the disclosure in hydrogen peroxide and a method of exposing the package of the disclosure in hydrogen peroxide mist, and it is preferable to effectively perform decontamination to cause exposure to hydrogen peroxide mist which causes less negative effects to the cell culture vessel.

The exposure of the package of the disclosure to the hydrogen peroxide mist is, for example, performed as follows. The decontaminating method of the disclosure includes a decontaminating step of decreasing concentration of hydrogen peroxide plasma or hydrogen peroxide vapor in an isolator, after exposing the package of the disclosure to hydrogen peroxide plasma or hydrogen peroxide vapor in the isolator. More specifically, in the decontaminating step, after putting the package of the disclosure in the isolator, for example, hydrogen peroxide is supplied into the isolator, and the package is exposed to gas in which the concentration of hydrogen peroxide plasma or hydrogen peroxide vapor is preferably from 10 to 1000 ppm, for 1 minute to 60 minutes. After that, hydrogen peroxide is discharged while introducing cleaned air into the isolator from the outside so that the concentration of hydrogen peroxide in the isolator becomes preferably equal to or smaller than 1 ppm preferably within 150 minutes, or hydrogen peroxide in the isolator is decomposed by using ultraviolet irradiation.

The present disclosure relates to the following one or plural embodiments.

[1] A packaging bag for a cell culture vessel used for sealing a cell culture vessel,

wherein when the packaging bag for a cell culture vessel sealing the cell culture vessel is accommodated in a closed space in which ordinary pressure is maintained, a temperature of 45° C. is maintained and the volume is 170 L, and the total amount of 70 mL of 6-mass % hydrogen peroxide aqueous solution is vaporized by applying ultrasonic vibration for 7 minutes in the closed space, and the hydrogen peroxide is decomposed by ultraviolet irradiation at a wavelength of 254 nm so that hydrogen peroxide concentration in the closed space becomes equal to or smaller than 1 mg/L within 90 minutes from the stop of the ultrasonic vibration, the amount of hydrogen peroxide adsorbed to the cell culture vessel is less than 3 ng/cm².

[2] The packaging bag for a cell culture vessel according to [1], in which a packaging material configuring the packaging bag is at least one type selected from a laminated film including a base film, an inorganic thin film layer provided on one surface side of the base film, and a sealant layer provided on another surface side of the base film, a laminated film including a base film and a sealant layer provided on one surface side of the base film, and a laminated film including a metal layer and a sealant layer provided on one surface side of the metal layer.

[3] The packaging bag for a cell culture vessel according to [2], in which a material of the base film is polyester or polyamide.

[4] The packaging bag for a cell culture vessel according to [2] or [3], in which the inorganic thin film layer is a vapor deposited film containing silicon oxide.

[5] The packaging bag for a cell culture vessel according to [2] or [3], in which the metal layer is a film containing aluminum.

[6] The packaging bag for a cell culture vessel according to any one of [1] to [5], in which the packaging bag for a cell culture vessel is a single-layered packaging bag or a multiple-layered packaging bag.

[7] The packaging bag for a cell culture vessel according to any one of [1] to [6], in which vapor permeability of the packaging material measured under the measurement conditions of 40° C. and 90% RH (relative humidity) based on JIS Z0222 is equal to or smaller than 0.2 g/(m²·24 hr).

[8] The packaging bag for a cell culture vessel according to any one of [1] to [7], in which oxygen permeability of packaging material measured under the measurement conditions of 25° C. and 80% RH (relative humidity) based on JIS K7126-2 is equal to or smaller than 2.0 mL/(m²·24 hr·MPa).

[9] The packaging bag for a cell culture vessel according to any one of [1] to [8], including a gas absorber in the bag.

[10] A package including: the packaging bag for a cell culture vessel according to any one of [1] to [9]; and the cell culture vessel sealed in the packaging bag for a cell culture vessel.

[11] A manufacturing method of a package including:

a step of sealing a cell culture vessel in the packaging bag for a cell culture vessel according to any one of [1] to [8]; and

a sterilization step of performing radiation sterilization with respect to the packaging bag for a cell culture vessel sealing the cell culture vessel.

[12] The manufacturing method of a package according to [11], in which the packaging bag for a cell culture vessel sealing the cell culture vessel is irradiated with a electron ray of 100 kGy/hr to 100 MGy/hr in the sterilization step.

[13] A manufacturing method of a package including:

a step of sealing a cell culture vessel in the packaging bag for a cell culture vessel according to [9]; and

a sterilization step of performing γ ray sterilization with respect to the packaging bag for a cell culture vessel sealing the cell culture vessel.

[14] A decontaminating method of a package including:

a step of performing hydrogen peroxide treatment with respect to a package manufactured by the manufacturing method of a package according to any one of [11] to [13]; and

in which hydrogen peroxide concentration in an atmosphere containing the hydrogen peroxide is decreased, after exposing the package to hydrogen peroxide, in the step of performing hydrogen peroxide treatment.

[15] The decontaminating method of a package according to [14],

in which the package is exposed to hydrogen peroxide vapor or hydrogen peroxide plasma, in the step of performing hydrogen peroxide treatment.

[16] The decontaminating method of a package according to [14] or [15],

in which hydrogen peroxide concentration in the atmosphere is decreased by decomposing hydrogen peroxide by ultraviolet irradiation with respect to the hydrogen peroxide vapor, in the step of performing hydrogen peroxide treatment.

[17] The decontaminating method of a package according to [14] or [15],

in which hydrogen peroxide concentration in the atmosphere is decreased by discharging hydrogen peroxide from the atmosphere, in the step of performing hydrogen peroxide treatment.

Hereinafter, the disclosure will be described based on the following examples and comparative examples, but the disclosure is not limited thereto.

### EXAMPLES

### Hydrogen Peroxide Permeability Test

(1) A polystyrene resin-made petri dish having a diameter of 90 mm (MS-13900 manufactured by Sumitomo Bakelite Co., Ltd.) was sealed in packaging bags of the following Examples 1 and 2 and Comparative Examples 1 and 2, and packaging bags containing the petri dish (package) were obtained.

(2) A H₂O₂ generator (MCO-HP-PJ manufactured by Panasonic Corporation) was connected in a CO₂ incubator (MCO-170AICUVH-PJ, volume of 170 L, manufactured by Panasonic Corporation), the packaging bags containing the petri dish obtained in (1) were put in the CO₂ incubator, 78 mL of hydrogen peroxide water (6-weight % hydrogen peroxide manufactured by Panasonic Corporation) was supplied to the H₂O₂ generator. After that, after increasing a temperature in the incubator to 45° C., hydrogen peroxide vapor is generated by using ultrasonic waves, and hydrogen peroxide vapor was sprayed to the packaging bags containing the petri dish in the atmosphere under the ordinary pressure at 45° C. The time of ultrasonic waves applied to hydrogen peroxide water was set as 7 minutes. After stopping the applying ultrasonic waves to hydrogen peroxide water, hydrogen peroxide was decomposed by immediately irradiating hydrogen peroxide vapor with ultraviolet rays at a wavelength of 254 nm for 90 minutes, to set hydrogen peroxide vapor concentration in the CO₂ incubator to be equal to or smaller than 1 mg/L. The processes of increasing a temperature to 45° C. and decomposition of hydrogen peroxide were performed by using a program of a device used in this example. After the irradiation of the ultraviolet rays, 8 mL of hydrogen peroxide has remained in the CO₂ incubator, and accordingly, 70 mL of hydrogen peroxide water was used in the spraying of hydrogen peroxide vapor.

(3) After that, the petri dish was extracted from the packaging bag, 3 mL of ultrapure water was put into the petri dish located on a horizontal surface, and hydrogen peroxide adsorbed to a bottom surface of the petri dish and some parts of an inner surface of the petri dish adjacent to the bottom surface (hereinafter, referred to as “surfaces to be cleaned”) was dissolved in the ultrapure water. The area of the surfaces to be cleaned is 58.5 cm².

(4) The ultrapure water obtained in (3) in which hydrogen peroxide is dissolved, was colored by using PACKTEST Hydrogen Peroxide (manufactured by Kyoritsu Chemical-Check Lab., Corp., WAK-H₂O₂, measurement range H₂O₂ 0.05 to 5 ppm, 4-Aminoantipyrene Method using Peroxidase) to measure absorbance of light at a wavelength of 550 nm. The obtained absorbance was converted into remaining hydrogen peroxide concentration [ppm] and hydrogen peroxide adsorption amount [ng/cm²] by using a separately drawn calibration curve, and the results were shown in Table 1.

Colony Forming Test

96 well plate (MS-8096F manufactured by Sumitomo Bakelite Co., Ltd.) was sealed in packaging bags in the following Examples 3 and 4 and Experimental Example 1 to obtain packages, and the packages were a target of gamma ray sterilization or electron beam sterilization. Then, after leaving the package at room temperature (25° C.) for 4 weeks, one NS-1 (mouse myeloma) cell and 0.1 mL of 10% fetal bovine serum-added MEM culture medium were put into each well, to cause culturing in the 5% CO₂ atmosphere at 37° C. for 7 days, and then, the number of wells where colony was formed due to proliferation of NS-1 cell was investigated and the results thereof were shown in Table 1.

Details of the packages, the packaging bags, and the packaging materials of Examples 1 to 4, Comparative Examples 1 and 2 and Experimental Example 1 are as follows.

### Example 1

A polyethylene sheet (thickness of 80 μm, weight average molecular weight of polyethylene), as a heat sealing layer, was laminated on TECHBARRIER HX (thickness: approximately 12 μm, manufactured by Mitsubishi Plastics, Inc.) obtained by forming a silica vapor deposited film on one surface of a PET film (12 μm) to obtain a packaging material for a packaging bag. A bag having a size of 145 mm×220 mm was manufactured by using this packaging material, one petri dish (MS-13900 manufactured by Sumitomo Bakelite Co., Ltd.) having a diameter of 90 mm was put therein, the bag is sealed to manufacture a package. Vapor permeability of the packaging material measured under the measurement conditions of 40° C. and 90% RH based on JIS Z0222 was 0.08 g/(m²·24 hr), and oxygen permeability thereof measured under the measurement conditions of 25° C. and 80% RH (relative humidity) based on JIS K7126-2 was 1.0 mL/(m²·24 hr·MPa).

### Example 2

A package was manufactured in the same manner as in Example 1, except for using a packaging material having the following configuration.

Heat sealing layer (polyethylene) 20 μm/aluminum foil 9 μm/polyethylene 15 μm/nylon 15 μm

### Example 3

A package was manufactured in the same manner as in Example 1, except for packaging a gas absorber (OxyCatch (registered trademark) manufactured by Kyodo Printing Co., Ltd.) 400 cm² in the packaging bag together with the cell culture vessel, and using 96 well plate (MS-8096F manufactured by Sumitomo Bakelite Co., Ltd.) as the cell culture vessel, instead of using the petri dish (MS-13900 manufactured by Sumitomo Bakelite Co., Ltd.) having a diameter of 90 mm. The obtained package was irradiated with γ rays. In order to set an abacterial level of the cell culture vessel as an abacterial level required for medical devices, a dose rate of γ rays was set as 1 kGy/h and an amount of irradiated radiation thereof was set as 19 kGy.

### Example 4

A package was manufactured in the same manner as in Example 1, except for using 96 well plate (MS-8096F manufactured by Sumitomo Bakelite Co., Ltd.) instead of the petri dish (MS-13900 manufactured by Sumitomo Bakelite Co., Ltd.) having a diameter of 90 mm. The obtained package was irradiated with electron beams. In order to set an abacterial level of the cell culture vessel as an abacterial level required for medical devices, a dose rate of electron beams was set as 1000 kGy/h and an amount of irradiated radiation thereof was set as 19 kGy.

### Comparative Example 1

A package was manufactured in the same manner as in Example 1, except for using a packaging material having the following configuration.

Heat sealing layer (polyethylene) 60 μm/nylon 25 μm

### Comparative Example 2

A package was manufactured in the same manner as in Example 1, except for using a packaging material having the following configuration.

Heat sealing layer (polyethylene) 50 μm/polyethylene terephthalate 12 μm

### Experimental Example 1

A package was manufactured in the same manner as in Example 1, except for using 96 well plate (MS-8096F manufactured by Sumitomo Bakelite Co., Ltd.) instead of petri dish (MS-13900 manufactured by Sumitomo Bakelite Co., Ltd.) having a diameter of 90 mm. The obtained package was irradiated with γ rays. In order to set an abacterial level of the cell culture vessel as an abacterial level required for medical devices, a dose rate of γ rays was set as 1 kGy/h and an amount of irradiated radiation thereof was set as 19 kGy.

TABLE 1

| | | | | | Comparative | Comparative | Experimental |
|---|---|---|---|---|---|---|---|
| | Example 1 | Example 2 | Example 3 | Example 4 | Example 1 | Example 2 | example 1 |
| Remaining | 0.006 | 0.017 | — | — | 2.16 | 2.53 | — |
| hydrogen | | | | | | | |
| peroxide | | | | | | | |
| concentration | | | | | | | |
| [ppm] | | | | | | | |
| Hydrogen | 0.32 | 0.87 | — | — | 113.7 | 133 | — |
| peroxide | | | | | | | |
| adsorption | | | | | | | |
| amount | | | | | | | |
| [ng/cm²] | | | | | | | |
| Colony | — | — | 52 | 33 | — | — | 0 |
| forming well | | | | | | | |
| number | | | | | | | |
| [number/96 | | | | | | | |
| well] | | | | | | | |

As shown in Table 1, when Examples 1 and 2 and Comparative Examples 1 and 2 are compared to each other, in Examples 1 and 2, the amount of hydrogen peroxide adsorbed to the cell culture vessel was significantly small, compared to Comparative Examples 1 and 2. Accordingly, in Examples 1 and 2, it is possible to expect prevention of negative effects of hydrogen peroxide to productivity of cells or cell aggregation, compared to Comparative Examples 1 and 2. According to “Report of Industrialization Advancement Business for regenerative medicines 2013 (Macular Degeneration, Homologous Ips Cell-Derived Retinal Pigment Epithelial Cell) p2 to p3, when an amount of hydrogen peroxide dissolved in a culture medium exceeds 0.5 ppm (mg/mL), a survival rate of iPS cells is significantly decreased. In order to set the amount of hydrogen peroxide dissolved in a culture medium to be 1/10 or less of 0.5 ppm (mg/mL), for example, it is necessary to set the amount of hydrogen peroxide adsorbed to the cell culture vessel to be equal to or smaller than 3 ng/cm², and when this absorption amount is used, it is considered that the effect to the survival rate of iPS cells is small. Therefore, in Examples 1 and 2, it is considered that the survival rate of iPS cells is higher than that in Comparative Examples 1 and 2.

The present disclosure is advantageous in research of human ES cell and a medical field such as regenerative medicine and the like, for example.

While preferred embodiments of the invention have been described and illustrated above, it should be understood that these are exemplary of the invention and are not to be considered as limiting. Additions, omissions, substitutions, and other modifications can be made without departing from the spirit or scope of the present invention. Accordingly, the invention is not to be considered as being limited by the foregoing description, and is only limited by the scope of the appended claims.

### EXPLANATION OF REFERENCES

1: package for cell culture vessel
2: cell culture vessel
3: hydrogen peroxide indicator
41, 51: base film
42: inorganic thin film layer
43, 52, 62: sealant layer
61: metal layer

What is claimed is:

1. A packaging bag for a cell culture vessel used for sealing a cell culture vessel,
wherein when the packaging bag for a cell culture vessel sealing the cell culture vessel is accommodated in a closed space in which ordinary pressure is maintained, a temperature of 45° C. is maintained and the volume is 170 L, and the total amount of 70 mL of 6-mass % hydrogen peroxide aqueous solution is vaporized by applying ultrasonic vibration for 7 minutes in the closed space, and the hydrogen peroxide is decomposed by ultraviolet irradiation at a wavelength of 254 nm so that hydrogen peroxide concentration in the closed space becomes equal to or smaller than 1 mg/L within 90 minutes from the stop of the ultrasonic vibration, the amount of hydrogen peroxide adsorbed to the cell culture vessel is less than 3 ng/cm².

2. The packaging bag for a cell culture vessel according to claim 1,
wherein a packaging material configuring the packaging bag is at least one type selected from a laminated film including a base film, an inorganic thin film layer provided on one surface side of the base film, and a sealant layer provided on another surface side of the base film, a laminated film including a base film and a sealant layer provided on one surface side of the base film, and a laminated film including a metal layer and a sealant layer provided on one surface side of the metal layer.

3. The packaging bag for a cell culture vessel according to claim 2,
wherein a material of the base film is polyester or polyamide.

4. The packaging bag for a cell culture vessel according to claim 2,
wherein the inorganic thin film layer is a vapor deposited film containing silicon oxide.

5. The packaging bag for a cell culture vessel according to claim 2,
wherein the metal layer is a film containing aluminum.

6. The packaging bag for a cell culture vessel according to claim 1,
wherein the packaging bag for a cell culture vessel is a single-layered packaging bag or a multiple-layered packaging bag.

7. The packaging bag for a cell culture vessel according to claim 1,
wherein vapor permeability of the packaging material measured under the measurement conditions of 40° C. and 90% RH (relative humidity) is equal to or smaller than 0.2 g/(m²·24 hr).

8. The packaging bag for a cell culture vessel according to claim 1,
wherein oxygen permeability of packaging material measured under the measurement conditions of 25° C. and 80% RH (relative humidity) is equal to or smaller than 2.0 mL/(m²·24 hr·MPa).

9. The packaging bag for a cell culture vessel according to claim 1, comprising:
a gas absorber in the bag.

10. A package comprising:
the packaging bag for a cell culture vessel according to claim 1; and
the cell culture vessel sealed in the packaging bag for a cell culture vessel.

11. A manufacturing method of a package comprising:
a step of sealing a cell culture vessel in the packaging bag for a cell culture vessel according to claim 1; and
a sterilization step of performing radiation sterilization with respect to the packaging bag for a cell culture vessel sealing the cell culture vessel.

12. The manufacturing method of a package according to claim 11,
wherein the packaging bag for a cell culture vessel sealing the cell culture vessel is irradiated with a electron ray in the sterilization step.

13. A manufacturing method of a package comprising:
a step of sealing a cell culture vessel in the packaging bag for a cell culture vessel according to claim 9 together with the gas absorber; and
a sterilization step of performing γ ray sterilization with respect to the packaging bag for a cell culture vessel sealing the cell culture vessel.
